(19) United States
(12) Patent Application Publication
(10) Pub. No.: US 2021/0322488 A1
(43) Pub. Date: Oct. 21, 2021

(54) Treatment Methodology for the Prevention and Control of Viral Infections

(71) Applicants: Yasser Dbouk, Beirut (LB); Zaher Dbouk, Beirut (LB); Mark Shaw, Ponte Vedra Beach, FL (US)

(72) Inventors: Yasser Dbouk, Beirut (LB); Zaher Dbouk, Beirut (LB); Mark Shaw, Ponte Vedra Beach, FL (US)

(21) Appl. No.: 17/232,301

(22) Filed: Apr. 16, 2021

Related U.S. Application Data
(60) Provisional application No. 63/010,937, filed on Apr. 16, 2020.

(51) Int. Cl. A61K 35/66; A61K 9/00; A61K 47/46; A61K 47/26

(52) U.S. Cl. CPC A61K 35/66; A61K 47/26; A61K 47/46; A61K 9/0043

(57) ABSTRACT

A treatment methodology for the prevention and control of viral infections by administering an anti-viral composition having organotrophic archaea microorganisms, a carrier substance and an activating liquid, characterized in that the archaea microorganisms, when present in human or animal, weaken or destroy a virus upon contact. The anti-viral composition is administered either intraorally or intranasally.

### BACKGROUND

This invention relates generally to human or animal treatment methodologies utilizing an anti-viral composition containing archaea to neutralize, weaken or eradicate a harmful virus, including the Novel Coronavirus (Covid19), HIV, influenza, etc., thereby preventing or controlling viral infections.

Archaea constitute a domain of single-celled organisms. These microorganisms lack cell nuclei and are therefore prokaryotes. Archaeal cells have unique properties separating them from the other two domains, Bacteria and Eukaryota. Archaea are further divided into multiple recognized phyla. Classification is difficult because most have not been isolated in the laboratory and have been detected only by analysis of their nucleic acids in samples from their environment. Archaea and bacteria are generally similar in size and shape, although a few archaea have very different shapes, such as the flat and square cells of Haloquadratum walsbyi. Despite this morphological similarity to bacteria, archaea possess genes and several metabolic pathways that are more closely related to those of eukaryotes, notably for the enzymes involved in transcription and translation. Other aspects of archaeal biochemistry are unique, such as their reliance on ether lipids in their cell membranes, including archaeols. Archaea use more energy sources than eukaryotes: these range from organic compounds, such as sugars, to ammonia, metal ions or even hydrogen gas. Salt-tolerant archaea (the Haloarchaea) use sunlight as an energy source, and other species of archaea fix carbon, but unlike plants and cyanobacteria, no known species of archaea does both. Archaea reproduce asexually by binary fission, fragmentation, or budding; unlike bacteria, no known species of Archaea forms endospores.

The first observed archaea were extremophiles, living in extreme environments, such as hot springs and salt lakes with no other organisms. Improved detection tools led to the discovery of archaea in almost every habitat, including soil, oceans, and marshlands. Archaea are particularly numerous in the oceans, and the archaea in plankton may be one of the most abundant groups of organisms on the planet.

Archaea are a major part of life on Earth. They are part of the microbiota of all organisms. In the human microbiota, they are important in the gut, mouth, and on the skin. Their morphological, metabolic, and geographical diversity permits them to play multiple ecological roles: carbon fixation; nitrogen cycling; organic compound turnover; and maintaining microbial symbiotic and syntrophic communities, for example.

No clear examples of archaeal pathogens or parasites are known. Instead they are often mutualists or commensals, such as the methanogens (methane-producing strains) that inhabit the gastrointestinal tract in humans and ruminants, where their vast numbers aid digestion. Methanogens are also used in biogas production and sewage treatment, and biotechnology exploits enzymes from extremophile archaea that can endure high temperatures and organic solvents.

Archaea exhibit a great variety of chemical reactions in their metabolism and use many sources of energy. These reactions are classified into nutritional groups, depending on energy and carbon sources. Some archaea obtain energy from inorganic compounds such as sulfur or ammonia (they are chemotrophs). These include nitrifiers, methanogens and anaerobic methane oxidizers. In these reactions one compound passes electrons to another (in a redox reaction), releasing energy to fuel the cell's activities. One compound acts as an electron donor and one as an electron acceptor. The energy released is used to generate adenosine triphosphate (ATP) through chemiosmosis, the same basic process that happens in the mitochondrion of eukaryotic cells.

Other groups of archaea use sunlight as a source of energy (they are phototrophs), but oxygen-generating photosynthesis does not occur in any of these organisms. Many basic metabolic pathways are shared among all forms of life; for example, archaea use a modified form of glycolysis (the Entner-Doudoroff pathway) and either a complete or partial citric acid cycle. These similarities to other organisms probably reflect both early origins in the history of life and their high level of efficiency.

Nutritional types in archaeal metabolism

| Nutritional | Source of | Source of | |
|---|---|---|---|
| type | energy | carbon | Examples |
| Phototrophs | Sunlight | Organic | Halobacterium |
| | | compounds | |
| Lithotrophs | Inorganic | Organic | Ferroglobus, |
| | compounds | compounds or | Methanobacteria or |
| | | carbon fixation | Pyrolobus |
| Organotrophs | Organic | Organic | Pyrococcus, |
| | compounds | compounds or | Sulfolobus or |
| | | carbon fixation | Methanosarcinales |

Some Euryarchaeota are methanogens (archaea that produce methane as a result of metabolism) living in anaerobic environments, such as swamps. This form of metabolism evolved early, and it is even possible that the first free-living organism was a methanogen. A common reaction involves the use of carbon dioxide as an electron acceptor to oxidize hydrogen. Methanogenesis involves a range of coenzymes that are unique to these archaea, such as coenzyme M and methanofuran. Other organic compounds such as alcohols, acetic acid or formic acid are used as alternative electron acceptors by methanogens. These reactions are common in gut-dwelling archaea. Acetic acid is also broken down into methane and carbon dioxide directly, by acetotrophic archaea. These acetotrophs are archaea in the order Methanosarcinales, and are a major part of the communities of microorganisms that produce biogas.

Other archaea use CO₂ in the atmosphere as a source of carbon, in a process called carbon fixation (they are autotrophs). This process involves either a highly modified form of the Calvin cycle or another metabolic pathway called the 3-hydroxypropionate/4-hydroxybutyrate cycle. The Crenarchaeota also use the reverse Krebs cycle while the Euryarchaeota also use the reductive acetyl-CoA pathway. Carbon fixation is powered by inorganic energy sources. No known archaea carry out photosynthesis. Archaeal energy sources are extremely diverse, and range from the oxidation of ammonia by the Nitrosopumilales to the oxidation of hydrogen sulfide or elemental sulfur by species of Sulfolobus, using either oxygen or metal ions as electron acceptors.

Organotrophic archaea use organic compounds, such as glucose or amino acids, as food sources to sustain growth and development. Organotrophic organisms obtain hydrogen or electrons from organic substances. Organotrophs may be either anaerobic or aerobic.

Viruses are divided into two major groups, enveloped and non-enveloped. Enveloped viruses, such as influenza, coronaviruses and many others, have an outer wrapping or envelope that comes from the infected cells as the virus is budding off. This envelope, along the matrix part (viral origin), carry what are called glycoproteins (HA for influenza, Spike for coronavirus, etc.), that play major roles in virus infection.

It is an object of this invention to provide a treatment methodology for the prevention and control of viral infections in humans and animals wherein a composition containing an effective amount of archaea is administered intraorally or intranasally.

### SUMMARY OF THE INVENTION

The invention is a treatment method for the prevention and control of viruses in humans or animals utilizing an anti-viral composition containing nonpathogenic archaea, more preferably a combination of different archaea microorganisms, and most preferably a combination of approximately at least 100 different archaea microorganisms. The archaea is combined with a solid carrier substance, such as powdered sugar or bentonite clay, and mixed with an activator liquid, such as water. The composition is administered to an animals or humans by oral ingestion, inhalation or spraying into the nostrils in order to prevent, control and eradicate viral infections by attacking the invading virus.

### DETAILED DESCRIPTION OF THE INVENTION

The invention is in general a treatment method to prevent and control viral infections in humans and animals though administration of an anti-viral composition containing nonpathogenic archaea, more preferably a combination of different archaea microorganisms, and most preferably a combination of approximately 100 different archaea microorganisms. The composition is administered to an animal or human by oral ingestion, inhalation, spraying into the nostrils, or coating the interior of the nostrils in order to prevent, control and eradicate viral infections by attacking the invading virus, such as infections caused by Covid19, HIV, influenza or the like. All or most of the archaea microorganisms in the composition are organotrophic.

Common viruses that are detrimental to humans or animals include influenza, the Human Immunodeficiency Virus (HIV), or the Novel Coronavirus (SARS-CoV-2), commonly referred to as Covid19. For ease of reference, the composition and methodology is discussed herein with reference to Covid19, but such targeted referencing is not meant to limit or restrict the viruses against which the composition and methodology may be utilized.

Covid19 is an enveloped virus, having a protective layer of sugar, fat, and lipids which helps to not only protect the virus and hide the virus from the human immune system, but also aids in its pathogenic properties once in the human body. The Covid19's fatty shield has spikes that are coated in sugars, known as glycans. These characteristics form the focus of the inventive methodology, which utilizes organotrophic archaea that feed on particular organic substances, to wit, sugar, fat and or lipids, to neutralize, weaken or eradicate the Covid19, thereby controlling or preventing viral infections in humans or animals.

A single organotrophic archaea microorganism, a combination of multiple organotrophic archaea microorganisms of different type, or most preferably, approximately at least 100 organotrophic archaea microorganisms of different type are combined with a carrier substance, such as powdered sugar or an ingestible clay, such as bentonite clay, with this mixture then combined with an activating liquid having a relatively neutral pH, such as water.

The archaea microorganisms are combined with or applied to the solid carrier substance to produce a mixture having billions of archaea microorganisms per gram. The mixture is mixed with an activator liquid, such as water. The composition is delivered to an animal or human by ingestion in liquid or solid form, or by inhalation, swabbing or spraying into the nostrils.

A representative and suitable product comprising the mixture of archaea and a carrier substance is available under the tradename ULTRA-ARCHAEA as sold by Ultratech International, Inc. of Jacksonville, Fla. This product in various mixtures contains from 5 to 90 billion archaea microorganisms per gram. The carrier substance is approximately 85% bentonite clay (a type of smectite clay formed through volcanic activity) and approximately 15% non-active, non-hazardous trace compounds, nutrients and other minerals (e.g., quartz, Cristobalite).

As a non-limiting example, from 0.5 to 5.0 grams of organotrophic archaea microorganisms is mixed with the carrier substance, most preferably a sugar or clay substance, the resulting mixture having approximately at least 1 billion archaea per gram, with a most preferred range being from 90-100 billion archaea per gram. The mixture is then combined with 25-100 ml of non-chlorinated water to create the composition. This composition is then brought into contact with the virus is various ways, such as intraorally or intranasally.

The composition may be ingested orally by swallowing the composition in liquid form prepared as previously discussed. The archaea and carrier substance may be pressed to form a compact body in pill or tablet form and swallowed along with water. The composition in powdered form may be sprinkled onto food and consumed. The liquid composition may be taken intranasally by snorting or spraying the composition into the nostrils or coating the nostrils with the composition using a swab or similar member. The composition could also be applied directly to a mask, thereby attacking the viruses on contact and providing a mechanism for inhalation of the composition throughout the period that the mask is worn.

Thus, the invention in various embodiments is a method of preventing and controlling viral infections in humans or animals comprising the steps of: providing an anti-viral composition comprising organotrophic archaea microorganisms, a carrier substance and an activating liquid, characterized in that the archaea microorganisms, when present in a human or animal, weaken or destroy a virus upon contact; administering the anti-viral composition to the human or animal in efficacious amount whereby viruses entering the human or animal come into contact with the anti-viral composition and are weakened or destroyed. Furthermore, the invention comprises such a method wherein the archaea microorganisms consist of different types of archaea microorganisms; wherein the archaea microorganisms consist of at least 100 different types of archaea microorganisms; wherein the carrier substance consists of sugar or bentonite clay; wherein the activating liquid is water; wherein the concentration of archaea microorganisms present in the combination of the archaea microorganisms and the carrier substance is from 2 to 100 billion per gram; wherein the step of administering the anti-viral composition to the human or animal is accomplished intraorally or intranasally; wherein the composition is swallowed; wherein the composition is sprayed into nostrils; wherein the composition is swabbed in nostrils; and/or wherein the step of administering the composition is repeated periodically.

In addition to delivering the archaea composition directly to a human or animal, it could be added to soapy water and detergents prior to using on hard surfaces to attack viruses on those surfaces.

It is believed that single or combinations of organotrophic archaea consume or diminish the protective fatty barrier layer of the virus, as they are adept consumers of sugars, fats, and lipids in all settings. By doing so, the archaea composition destroys the virus or weakens the protective layer of the virus, allowing it to more easily be attacked and neutralized from the body via medication or anti-bodies, both natural or synthetic.

The composition can be provided in the form of and used as both a preventative medicine and treatment medicine. For instance, the composition could be dosed once every three to six weeks on a regular basis as a preventative. Alternatively, or in addition to, the composition could be dosed prior to or immediately after possible exposure to a virus, such as would be more likely to occur in large gatherings, during air travel, etc.

It is understood that equivalents and substitutions for certain elements and steps described above may be obvious to those of skill in the art, and therefore the scope and definition of the invention is to be as set forth in the following claims.

We claim:

1. A method of preventing and controlling viral infections in humans or animals comprising the steps of:
providing an anti-viral composition comprising organotrophic archaea microorganisms, a carrier substance and an activating liquid, characterized in that the archaea microorganisms, when present in a human or animal, weaken or destroy a virus upon contact;
administering the anti-viral composition to the human or animal in efficacious amount whereby viruses entering the human or animal come into contact with the anti-viral composition and are weakened or destroyed.

2. The method of claim 1, wherein the archaea microorganisms consist of different types of archaea microorganisms.

3. The method of claim 1, wherein the archaea microorganisms consist of at least 100 different types of archaea microorganisms.

4. The method of claim 1, wherein the carrier substance consists of sugar or bentonite clay.

5. The method of claim 1, wherein the activating liquid is water.

6. The method of claim 1, wherein the concentration of archaea microorganisms present in the combination of the archaea microorganisms and the carrier substance is from 2 to 100 billion per gram.

7. The method of claim 1, wherein the step of administering the anti-viral composition to the human or animal is accomplished intraorally or intranasally.

8. The method of claim 7, wherein the composition is swallowed.

9. The method of claim 7, wherein the composition is sprayed into nostrils.

10. The method of claim 7, wherein the composition is swabbed in nostrils.

11. The method of claim 1, wherein the step of administering the composition is repeated periodically.

12. The method of claim 3, wherein the concentration of archaea microorganisms present in the combination of the archaea microorganisms and the carrier substance is from 2 to 100 billion per gram.

13. The method of claim 12, wherein the step of administering the anti-viral composition to the human or animal is accomplished intraorally or intranasally.

14. The method of claim 4, wherein the concentration of archaea microorganisms present in the combination of the archaea microorganisms and the carrier substance is from 2 to 100 billion per gram.

15. The method of claim 14, wherein the step of administering the anti-viral composition to the human or animal is accomplished intraorally or intranasally.

16. The method of claim 15, wherein the archaea microorganisms consist of at least 100 different types of archaea microorganisms.
